(19) United States
(12) Patent Application Publication
(10) Pub. No.: US 2020/0095551 A1
(43) Pub. Date: Mar. 26, 2020

(54) METHODS, SYSTEMS, FACTORS, AND MEDIA FOR REDUCTION OF CELLULAR STRESS AND REACTIVE OXYGEN SPECIES

(71) Applicant: LONGBOAT AMNIOTICS AB, Lund (SE)

(72) Inventors: Niels-Bjarne Woods, Furuland (SE); Roger Emanuel RÖNN, LUND (SE); Carolina GUIBENTIF, LUND (SE); Shobhit SAXENA, LUND (SE)

(21) Appl. No.: 16/494,662

(22) PCT Filed: Mar. 17, 2017
(86) PCT No.: PCT/US2017/023090

§ 371 Date: Sep. 16, 2019

(51) Int. Cl. C12N 5/0789; C12N 5/074

(52) U.S. Cl. CPC C12N 5/0647; C12N 5/0607; C12N 2500/02; C12N 2506/02; C12N 2501/125; C12N 2501/145; C12N 2501/26; C12N 2500/38

(57) ABSTRACT

The present invention relates to methods, systems, factors, and media for the reduction of cellular stress and reduction of the amount reactive oxygen species. Embodiments of the invention reduce cellular stress by treating cells with a variety of molecules, such as certain inhibitors. Some embodiments may reduce the amount of reactive oxygen species in cell media through the use of scavengers.

### CROSS-REFERENCE TO RELATED APPLICATIONS

This application is a U.S. national phase application of PCT/US2017/023090, filed Mar. 3, 2017. The disclosure of this prior application is hereby incorporated by reference in its entirety.

### STATEMENT REGARDING PRIOR DISCLOSURE BY THE INVENTOR OR A JOINT INVENTOR

Part of the present invention was disclosed by the inventors in Woods et al., Reactive Oxygen Species Impair the Function of CD90+ Hematopoietic Progenitors Generated from Human Pluripotent Stem Cells,” Stem Cells 35(1): 197-206. (24 Oct. 2016, online date 18 Sep. 2016).

### BACKGROUND OF THE INVENTION

### Field of the Invention

This application describes embodiments of methods, systems, factors and media for the reduction of cellular stress and/or the presence of reactive oxygen species (ROS) during generation of blood cells.

### Description of the Related Art

Methods for isolating cells, reprogramming cells, generating pluripotent and multipotent cells, tissues and organs, are needed for a variety of therapeutic applications, including personalized and regenerative medicine. A great variety of human stem cells and other cell types are known and characterized, including embryonic stem cells, isolated during early embryonic development, and somatic stem cells such as adult mesenchymal stem/stromal cells and hematopoietic stem cells. Somatic cells can also be reprogrammed into more primitive states of its respective lineage or into cells of a different lineage (even a differing germ layer derivative), or into cells with a specific desired function, or may differentiate into multiple types of different cells.

For example, human umbilical cord blood from newborn infants has been used as a source of hematopoietic stem cells for transplantation to patients with hematological disorders and malignancies for decades, due to the high proportion of blood stem cells present in the material. As such, there are currently hundreds of thousands of cord blood samples stored around the world in publicly and privately financed storage banks, ready to be used upon request. Cord blood cells can also be reprogrammed into pluripotent stem cells and differentiated to many of cell types. In another example, cells may be isolated from amniotic fluid, the aqueous medium that surrounds, protects, and aids in the fetal development, for example, by providing a mechanical barrier, providing growth factors, and aiding lung development by filling developing spaces in the lung to define what will become permanent air spaces. Further information regarding methods and apparatuses for the collection and isolation of cells may be found in U.S. Pat. Pub. 2016/00030489, filed Sep. 14, 2015, and U.S. Pat. Pub. 2016/0068815, filed Sep. 14, 2015, both of which are hereby incorporated by reference in their entireties.

Available approaches and apparatuses for collecting, extracting, and isolating biological components, including cellular material, umbilical cord blood, and cells found therein, have not been entirely satisfactory, for example, in their safety, avoidance of contamination (e.g., air contamination) of collected material, cell yield, efficiency, and/or ability to avoid destruction of components. Most recently, it was shown that even short term exposure to air during collection and laboratory processing of hematopoietic stem cells had deleterious effects on their function.

De novo hematopoietic stem, and progenitor cells can be generated in vitro from a number of methods: via pluripotent stem cell differentiation systems, cellular reprogramming towards hematopoietic cells or precursors, and induction of hematopoietic stem cell precursors by support cells and growth factors. Pluripotent stem cells (embryonic stem cells and induced pluripotent stem cells) may differentiate using systems that mimic embryonic/fetal development of hematopoietic stem cells and/or using factors guide or enhance differentiation towards the specific developmental stages towards hematopoietic stem cells, and indeed all other means of producing hematopoietic cells, requires controlled conditions that regulate numerous specific developmental factors spatially and temporally. For example BMP4 (Bone morphogenetic protein 4) is required to efficiently specify mesodermal lineage from pluripotent stem cells within 3 days of differentiation form pluripotent stem cells. Another example is that retinoic acid signaling is required in the development of hematopoietic stem cells as they emerge. While other factors have been identified, there are likely still numerous additional factors which have not yet been identified that play a role in generating blood. Moreover, the de novo generation of blood in vivo, and the generation of blood in vitro will also likely differ as developmental programs cannot yet be exactly mirrored in vitro, but may also allow for directed differentiation of starting cell materials for greater frequency and efficiency of blood cell generation in vitro.

In vivo during development of de novo blood, the levels of reactive oxygen species have not been studied nor is it understood how de novo hematopoietic cells, or the environment surrounding them (e.g. their niche), regulates reactive oxygen species. However, it is documented that the levels of ROS are relatively low in hematopoietic stem cells and progenitors when freshly isolated from their traditional sources (bone marrow, cord blood, and mobilized peripheral blood). Interestingly, upon expansion culture, these human hematopoietic stem cells show increased levels of ROS, and severely compromised repopulating activity in transplantation. One study showed that reducing the concentration of ROS in culture, such as by using an antioxidant NAC, could help preserve the repopulating activity of the cells. Most notably, the effects of ROS on the development of de novo hematopoietic stem cells and progenitors and their precursors in vitro has not been studied.

Some of the direct effects of elevated ROS levels are increased cellular stress and oxidative damage to DNA, proteins and lipids. The oxidative damage to DNA leads to accumulation of mutations and ultimately to apoptosis of the cell. Adult hematopoietic stem cells are sensitive to elevated ROS levels which can be generated in vitro by cellular proliferation and metabolic activity. ROS levels are also affected by extracellular conditions such as oxygen levels, and necrotic tissue fall out. ROS can also result from an inflammatory response, endoplasmic reticulum stress, and the apoptosis response. Accordingly, there is a need for improved methods, systems, and media for the generation of cells under conditions of lowered cellular stress and lowered concentrations of ROS.

### SUMMARY OF THE INVENTION

Embodiments of the present invention relate to methods, systems, and media for reducing cellular stress and/or the presence of ROS. It will be understood by one of skill in the art that the cells and ROS described herein are not limited to a specific type of cell or ROS unless otherwise specified.

In certain embodiments, a method of mediating cellular stress during transition of a cell into another cell type, comprises:

culturing said cell under conditions that lead to said transition of said cell while carrying out two or more of the following:
reducing the available oxygen in a medium surrounding the cell;
treating the cell with an antioxidant;
inhibiting a cellular stress response pathway of the cell;
inhibiting an innate immune response of the cell; and
activating a cellular pathway that reduces the concentration of intracellular reactive oxygen species.

In some embodiments, a method of mediating cellular stress in a human blood precursor cell during transition of said precursor cell into a blood cell, comprises:

culturing said human blood precursor cell under conditions that lead to said transition of said precursor cell into a blood cell while carrying out one or more of the following:
reducing the available oxygen in a medium surrounding the human blood precursor cell;
treating the human blood precursor cell with an antioxidant;
inhibiting a cellular stress response pathway of the human blood precursor cell;
inhibiting an innate immune response of the human blood precursor cell; and
activating a cellular pathway that reduces the concentration of intracellular reactive oxygen species.

In particular embodiments, the transition comprises differentiation of the cell to a more committed cell type. The transition may comprise a conversion of the cell into an induced pluripotent stem cell. Activating a cellular pathway that reduces the concentration of intracellular reactive oxygen species may comprise treating the cell with a cAMP signaling activator. The cAMP signaling activator may comprise Forskolin or IBMX. In certain embodiments, reducing the available oxygen in a medium surrounding the cell comprises placing the cell in a hypoxic environment.

In certain embodiments, the antioxidant comprises a component selected from the group consisting of ascorbic acid, citric acid, vitamin E, selenium, melatonin, NAC, glutathione, thioredoxin, nicotinamide adenine dinucleotide phosphate, Superoxide dismutase, Catalase, and Glutathione peroxidase, and Citric acid monohydrate. The concentration of ascorbic acid may be about 0.011-0.55 mg/ml, while the concentration of citric acid may be about 0.115-1.15 mg/ml. Inhibiting the cellular stress response pathway may comprise inhibiting mitochondrial p53 mediated apoptosis. Inhibiting the cellular stress response pathway may comprise inhibiting p38 mediated senescence. In certain embodiments, inhibition of the p38 mediated senescence comprises treating the cell with LY2228820 at a concentration range of about 20-500 nM. Inhibiting the cellular stress response pathway may comprise inhibiting endoplasmic reticulum stress. In certain embodiments, nhibiting the cellular stress response pathway comprises inhibiting non-mitochondrial calpain mediated stress. Inhibiting the innate immune response may comprise inhibiting myeloperoxidase production with a myeloperoxidase inhibitor. In certain embodiments, the myeloperoxidase inhibitor comprises 4-ABAH at a concentration range of about 100 μM.

Inhibiting the cellular stress response pathway may comprise inhibiting mitochondrial p53 mediated apoptosis with pfilthrin-μ. In certain embodiments, inhibiting the cellular stress response pathway comprises inhibiting non-mitochondrial calpain mediated apoptosis of the cell. Inhibiting non-mitochondrial calpain mediated apoptosis of the cell may comprise treating the cell with MDL28170 at a concentration range of about 0.5-25 μM. In certain embodiments, reducing the available oxygen for a cell comprises reducing the amount of oxygen in a culture system to about 4%. in embodiments, the cell may be an induced pluripotent stem (iPS) cell.

In embodiments, a medium for the de novo generation of human blood cells may comprise two or more of the following:
an antioxidant;
an inhibitor of mitochondrial p53 mediated apoptosis;
an inhibitor of non-mitochondrial calpain mediated apoptosis;
an inhibitor of endoplasmic reticulum stress;
an innate immune response inhibitor;
an inhibitor of p38 mediated senescence; and
a cAMP signaling pathway activator.

### BRIEF DESCRIPTION OF THE DRAWINGS

Other features and advantages of the present invention will be apparent from the following detailed description of the invention, taken in conjunction with the accompanying drawings of which:

FIG. 1 illustrates an embodiment of a method for the reduction of cellular stress and/or ROS.

FIGS. 2A-F depict the results of experiments utilizing an embodiment of a method showing that elevated ROS levels lead to reduced functionality of hPSC (human pluripotent stem cell)-Derived Hematopoietic Progenitors.

FIGS. 3A-F depict experimental results of an embodiment of a method showing colony forming capacities of ROSlo and ROShi hPSC-derived hematopoietic progenitors.

FIGS. 4A-G depict a drawing of an embodiment of an experimental method and accompanying results of experiments utilizing an embodiment of a method used to demonstrated that a combination of ROS-reducing strategies lead to increased generation of ROSlo CD90+ hematopoietic progenitors.

FIGS. 5A-C depict experimental results of an embodiment of a method showing that ROS reduction increases the generation of hPSC-derived CD90+ hematopoietic progenitor cells with high growth capacity.

FIGS. 6A-D depict experimental results of an embodiment of a method showing that ROS reduction preserves the functionality of cd90+ hematopoietic progenitor cells, without affecting endothelial cells.

FIGS. 7A-D depict experimental results of an embodiment of a method to demonstrate that cAMP activation reduces intracellular levels of ROS in pluripotent stem cell differentiation cultures.

### DETAILED DESCRIPTION OF THE PREFERRED EMBODIMENTS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. In another example, a description of a range in weeks also includes disclosure of the days between the week endpoints. This applies regardless of the breadth of the range.

As used herein, the term “safe” can be understood to refer to any method or apparatus which poses no significant risk of maternal and/or fetal harm.

As used herein, “isolated,” when used to describe a cell or cells, refers to a cell or cells that have been separated from their natural environment, including by separation from the subject from which the cell is derived, e.g., a patient, and/or by separation from one or more other components of the natural environment, such as debris, tissue, tissue aggregates, and other cells.

As used herein, “fetal” is used to describe the property of a cell or other material derived from a developing mammal, such as a human, after the embryonic stage and before birth. As used herein, “infant” is used to describe the property of a cell or other material derived from a newborn or young mammal, from birth to one year of age, including premature infants and newborns.

As used herein, “pluripotent” refers to the ability of a cell to differentiate into cell types of any of the three germ layers, for example endoderm, mesoderm, and ectoderm. “Multipotent” refers to the ability of a cell to differentiate into cells more than 2 lineages, but a limited number of lineages.

Precursor is a term used to describe a cell or a reprogrammed cell that is developmentally upstream of the desired cell. For example, a hematopoietic precursor of blood may include hemogenic endothelium.

In vitro derived hematopoietic cells including hematopoietic stem cells should be understood as cells derived from a differentiation process, or reprogramming event, or from an induction system using certain factors or cells with certain properties, that converts a non-hematopoietic stem or progenitor cell into a hematopoietic stem or progenitor cell. Typically this is exemplified by endothelial to hematopoietic transition whereby in vivo and in vitro it has been determined that the precursor of hematopoietic cells is an endothelial cell which by down regulation of an endothelial transcription program and upregulation of a hematopoietic transcriptional program the endothelial cell transitions to blood. The processes of generating hematopoietic stem or progenitor cells can conceivably be improved by combining the methods described above where the hematopoietic stem cell production method includes aspects of all or a number of the known methods for the differentiation of hematopoietic precursors, reprogramming cells to get specific precursors or HSCs themselves, and induction of hematopoietic stem cells from the precursors. All such combinations of methods and any future method of generating blood are within the scope of this disclosure as the overriding issue facing any in vitro factor based generation of de novo blood will have improved efficiency of hematopoietic stem cells generation and function by the reduction of ROS and cellular stress during the generation process. Novel or established factors may be used to generate blood when traditionally harvested hematopoietic stem cells from donors are be exposed to growth factors or reprogramming factors allowing them to be expanded by for example reprogramming into cells with expansion ability, and/or into precursors of hematopoietic stem cells which would then allow generation of expanded numbers of de novo hematopoietic stem cells. Again ROS and stress management will be required to increase the efficiency of generation of functional hematopoietic stem cells using these methods.

The phrase “stem cell(s)” may be used throughout the specification. It will be understood by one of skill in the art that “stem cell(s)” may refer to adult stem cells or embryonic stem cells and human or animal stem cells. For example, such stem cells may include induced pluripotent stem (iPS) cells that have been generated from any adult cell types, including skin, fibroblasts and other cells and tissues. Masip et al., 2010, Mol Hum Reprod 16(11): 856-868; Takahashi and Yamanaka, 2006, Cell 126(4): 663-676; Yu et al., 2007, Science 318(5858): 1917-1920. A variety of stem cells are currently used therapeutically or evaluated for use in clinical trials, including somatic cells, such as mesenchymal stem/stromal cells, and hematopoietic stem cells, e.g., for use in neurological and hematological disorders, respectively. For the purposes of this document mesenchymal stem cell and mesenchymal stroma cell can be used interchangeably.

The phrase “reprogrammed somatic cell” may be used throughout the specification. It will be understood by one of skill in the art that “reprogrammed somatic cell” is not limited to a particular type of somatic cell, but rather may refer to any type of somatic cell. Takahashi and Yamanaka first described reprogramming technologies to “reprogram” or “de-differentiate” somatic cells into a pluripotent/embryonic like state, or to directly “reprogram” somatic cells into another cell lineage type. Takahashi and Yamanaka, 2006, Cell 126(4): 663-676. For example, reprogrammed somatic cells may refer to reprogramed cells from epithelial, connective, nervous, muscle tissues and/or from blood, such as umbilical cord blood. For example, cord blood derived endothelial progenitor cells are suitable for reprogramming. Non somatic cells (e.g. germ cells) may also be reprogrammed and their use with regards to derived cells relevant for this invention, should be considered equivalent to somatic reprogrammed cells.

In the following description of certain embodiments provided here, reference is made to the accompanying drawings which form a part hereof, and in which it is shown by way of illustration specific embodiments in which the invention can be practiced. It is to be understood that other embodiments can be used and structural changes can be made without departing from the scope of the invention.

Stem cells, for example, adult hematopoietic stem cells (HSCs), both human and animal, have been demonstrated to be highly sensitive to increased levels of ROS, where ROS-mediated oxidative damage impairs the self-renewal and long-term (LT) engraftment capacity of these cells. ROS is a collective term for oxygen containing molecules that, due to unpaired valence shell electrons, are highly reactive, causing oxidative damage to components of the cell including DNA, proteins, and lipids 10, and results in cell cycle arrest, premature senescence, or apoptosis. Accumulation of oxidative damage to biomolecules contributes to phenotypes and diseases associated with aging and cancer, as argued by the free radical theory of aging. In normal cells, the vast majority of ROS originates from the mitochondria as a by-product of cellular metabolism through oxidative phosphorylation. The intracellular levels of ROS are regulated through an intricate system of factors including available nutritional antioxidants (such as vitamin C, vitamin E, and selenium), reducing co-factors and peptides (glutathione, thioredoxin, and nicotinamide adenine dinucleotide phosphate), and antioxidant enzymes (Superoxide dismutase, Catalase, and Glutathione peroxidase).

Additional mechanisms have also been shown to influence cellular ROS, including active ROS detoxification by neighboring cells in the niche, the distance of the cell to the microvasculature proliferative activity of the cells, the release of ROS in the niche by innate immune cells. At controlled physiological levels, cells depend on ROS for oxidative turnover of proteins required for signal transduction, with ROS as a central mediator in signaling pathways involved in proliferation, differentiation, and quiescence. Thus, there may be a cellular “redox window” where an appropriate ROS level is required for physiological cellular function, while increased ROS can contribute to cellular dysfunction and pathological conditions. Depending on the concentration of oxygen, ROS can result from spontaneous oxidation. Furthermore, effector cells of innate immunity, such as granulocytes, can enzymatically release ROS into the extracellular space. In addition to being a cause of stress, ROS has also been reported to be produced in cells intended for apoptosis or senescence, with active ROS generation identified as downstream of p53 activation, Endoplasmic Reticulum stress, and by the p38-mediated stress response, indicating that ROS is a central and shared feature between the various classical pathways of stress signaling. It has been demonstrated that primary (non-cultured) hematopoietic progenitors from cord blood rapidly transit from a ROSlo (ROS low) to a ROShi (ROS high) phenotype when cultured in vitro, leading to impairment in LT engraftment capacity of HSCs. Thus, the physiological ROSlo phenotype is a primary requisite for the functionality of adult HSCs. Given that hPSC-derived hematopoietic cells have persistently demonstrated proliferative capacity deficits compared to adult and neonatal counterparts (bone marrow and cord blood progenitors), hPSC-derived hematopoietic stem cells (HSCs) are functionally impaired as a direct result of the elevated ROS levels and its associated consequences to cell proliferation

As described above, in vitro generation of de novo blood from pluripotent stem cells results in blood cells that show significantly elevated levels of intracellular ROS. Elevated levels of intracellular ROS are indicative of cellular stress, an undesirable condition for proper cell generation and growth. Elevated ROS has been shown to increase cellular DNA damage, produce a lower frequency of more functional stem cells, reduce colony forming capacity, and/or reduce the proliferative capacity of the stem cells. Additionally, for example, HSCs are well-known to lose their LT engraftment capacity when cultured in vitro, and it has been reported that cells with a low level of ROS, a characteristic feature of non-cultured and functional HSCs, will rapidly transit towards a ROShi phenotype upon sub-culture.

Additionally, further cell types, such as stem cells and reprogrammed somatic cells, may be negatively affected by elevated levels of ROS. Therefore, disclosed below and throughout the specification are methods, systems, and media for the reduction of cellular stress and/or the amount of ROS in a cell culture medium, containing cells such as stem cells and/or reprogrammed somatic cells.

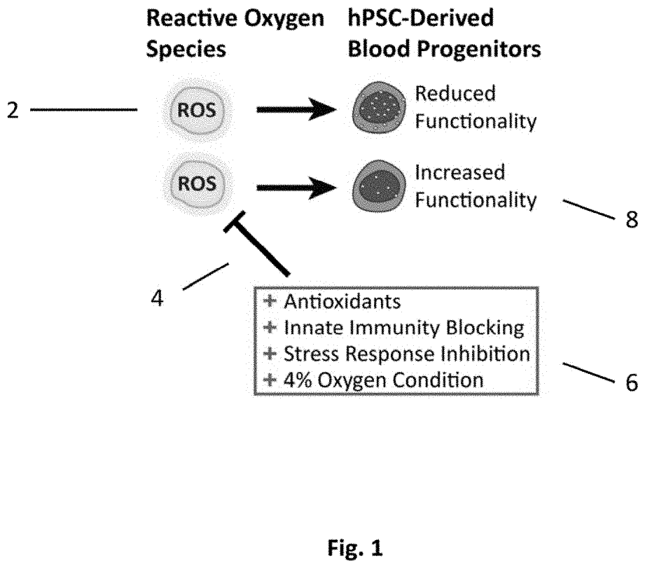

### The Methods, Systems, Factors, and Media of FIG. 1

FIG. 1 illustrates an embodiment of a method for reducing the level of ROS and/or cellular stress in a cell culture medium, the cell culture medium containing stem cells and/or reprogrammed somatic cells. As will be understood by one of skill in the art, stem cells and/or reprogrammed somatic cells differentiate over time while in culture. Therefore the different elements of the method(s) described herein this section and elsewhere in the specification may be applied at different times during the cell culture. For example, at about: 0 hours, 2 hours, 12 hours, 24 hours, 2 days, 4 days, 8 days, 10 days, 12 days, 14 days, 18 days or more than 18 days.

Returning to FIG. 1, in the untreated condition 2, the presence of high ROS may reduce the functionality of cells, such as stem cells or reprogrammed somatic cells. However, in certain embodiments, treatment 4 of cells and cell medium containing ROS with different factors and/or conditions 6, may lead to increased functionality 8 of the cells. In some embodiments, the method may include treating the cell medium with antioxidants, reducing the innate immune (inflammatory) response, cellular stress response pathway inhibition, and reduction in the concentration of oxygen, for example down to about 4%. In some embodiments, the concentration of oxygen may be about 0.5-10%, 1-6%, or 2-4%. In certain embodiments, the concentration of oxygen may be controlled by use of a hypoxic incubator or by other suitable means. In embodiments, the method may further include additional cell or medium treatments, such as upregulating genes that provide additional protection against stress, such as oxidative stress. In certain embodiments, the method may further include activating a cellular pathway that reduces the concentration of intracellular reactive oxygen species comprises (for example, via a cAMP signaling activator). It will be understood by one of skill in the art that there may be a synergistic effect of combining the elements described above or elsewhere in the specification. In certain embodiments, combining two of the method elements, three of the method elements, four of the method elements, and so forth may be beneficial. High levels of ROS and stress may lead to a vicious circle of events that causes damage to the cell leading to more stress response and leading to more ROS. Therefore, use of multiple method elements may combine to have a greater effect.

In certain embodiments, treatment of stem cells or reprogrammed somatic cells, such as hematopoietic cells, with the methods described herein this section or elsewhere in the specification may induce an increase in the efficiency of generating more functional cells. For example, in the case of hematopoietic cells, more primitive hematopoietic cells with CD90+ phenotype with proliferative ability may be generated. In certain embodiments, the more functional cells will be generated with an increased efficiency of about at least 5 fold, at least 10 fold, at least 15 fold, at least 20 or 21 fold, at least 25 fold, at least 30 fold, or more than a 30 fold increase in the generation of the more functional cells.

In certain embodiments, the antioxidants may comprise ascorbic acid (vitamin C), vitamin E, citric acid, selenium, N-acetylcysteine (NAC), melatonin, reducing co-factors and peptides Garcinol, glutathione, glutathione, thioredoxin, nicotinamide adenine dinucleotide phosphate, antioxidant enzymes such as superoxide dismutase, catalase, glutathione peroxidase, DPI, apocynin, NAC, MnTMPyP, gp91ds-tat peptide, and MitoTEMPO. In some embodiments, the antioxidant treatment may include a cocktail of the antioxidants described above. Antioxidants may be used at various concentrations, for example, about 0.01-1 mg/ml, about: 0.05-0.75 mg/ml, 0.1-0.5 mg ml, or about 0.35 mg/ml. In embodiments the amount of ascorbic acid may be approximately 0.378 mg/ml. In certain embodiments, the amount of citric acid may range from about: 0.01-2 mg/ml, 0.05-1.2 mg/ml, 0.1-0.8 mg/ml, about 0.125-0.6 mg/ml, 0.15-0.2 mg/ml or about 0.158 mg/ml. In certain embodiments, the concentration of Garcinol may be from about 0.1-10 μM (micromolar), 0.2-5 μM, 0.5-2 μM, or about 1 μM. NAC may be provided at concentrations of about 10-500 μM, 50-150 μM, or about 100 μM. Glutathione may be provided at concentrations of about 1-20 μM, 2-10 μM, or about 5 μM. Melatonin may be provided at concentrations of about 1-40 μM, 10-30 μM, or about 20 μM.

In certain embodiments, the cellular stress response may be inhibited via inhibition of pathways related to mitochondrial p53 mediated apoptosis, non-mitochondrial calpain mediated apoptosis, p38 mediated senescence, iron induced oxidative stress, and/or endoplasmic reticulum stress. The inhibition pathways described above and elsewhere in the specification may be inhibited individually, all at once, or in any possible combination of the above, for example, by inhibiting both mitochondrial p53 mediated apoptosis and p38 mediated senescence but not the other pathways.

In some embodiments, mitochondrial p53 mediated apoptosis may be inhibited by pfilthrin-μ. Pfilthrin-μ may be used in concentrations such as 1-20 μM, 5-15 μM, or about 10 μM. In embodiments, non-mitochondrial calpain mediated apoptosis may be inhibited by MDL28170. MDL28170 may be provided to the culture at concentrations of about 1-25 μM, 2-15 μM, 3-10 μM, or about 5 μM. In some embodiments, PD150606, SJA6017, ABT-705253, and SNJ-1945, and AK275 may be used instead of or in combination with MDL28170 at those same concentrations. Mitochondrial mediated apoptosis may be inhibited by the addition of Tauroursodeoxycholic acid (TUDCA) to the culture at a concentration of about 10-300 μM, 20-200 μM, 30-100 μM, or about 60 μM

In certain embodiments, the cellular stress response may be inhibited by inhibiting p38-mediated senescence, for example through use of p38 MAPK inhibitor LY2228820 at a concentration of about 10-1000 nM, 100-800 nM, 200-600 nM, or about 500 nM. in some embodiments, Acumapimod Bakuchiol, Bakuchiol BMS-582949 hydrochloride, Chelerythrine Chloride, Dehydrocorydaline chloride, Doramapimod, GNE-495, Losmapimod, Pamapimod, PH-797804, R1487 (Hydrochloride), SB 202190, SB 203580, SB203580 (RWJ 64809), SB 203580 hydrochloride, SB 239063, and/or SB 242235 may be used in place of or in combination with LY2228820.

In certain embodiments, iron-induced oxidative stress may be inhibited by Deferoxamine, an iron chelator. The concentration of Deferoxamine may be from about 0.1-10 μM, 0.2-5 μM, 0.5-2 μM, or about 1 μM.

In some embodiments, the inflammatory response may be reduced by blocking myeloperoxidase production and/or release. Myeloperoxidase may be blocked by addition of 4-amino benzoic acid (4-ABAH). 4-ABAH may be provided to the cell culture at a concentration of about 10-200 μM, 50-150 μM, or about 100 μM. In embodiments, 4-ABAH may be added to the culture at 8 days and onward or at any other time such as disclosed herein this section or elsewhere in the specification.

In certain embodiments, SRT1720 may be used in the method to upregulate genes protective of oxidative stress. SRT1720 may be utilized at a concentration of about: 0.1-10 μM, 0.2-5 μM, 0.5-2 μM, or about 1 μM. In some embodiments, Resveratrol (3,5,4′-trihydroxy-trans-stilbene), metformin, Oxaloacetate, SRT1720, SRT2104, SRT2379, Oxazolo[4,5-b]pyridines derivative, Imidazo[1,2-b]thiazole derivative, or 1,4-Dihydropyridine (DHP) derivatives may be used instead of or in combination with SRT1720 at the same or similar concentrations.

In embodiments, Rapamycin may be used in the method to regulate cell growth metabolism, therefore regulating oxidative stress. Rapamycin may be added to the cell culture at a concentration of approximately 0.000001-10 μM, 0.001-5 μM, 0.01-1 μM, or about 0.1 μM. Rapamycin may be added to the cell culture at various times during differentiation, for example at 8 days for 2-3 consecutive days.

In certain embodiments, CHIR 99021 may be used in the method to as a Wnt signaling pathway activator to mediate mesodermal and hematopoietic signaling. CHIR 99021 may be used in concentrations of 0.1-10 μM, 0.2-5 μM, 0.5-2 μM, or about 1 μM. In some embodiments, BIO (also a Glycogen Synthase Kinase 3 inhibitor (see Tocris) may be used.

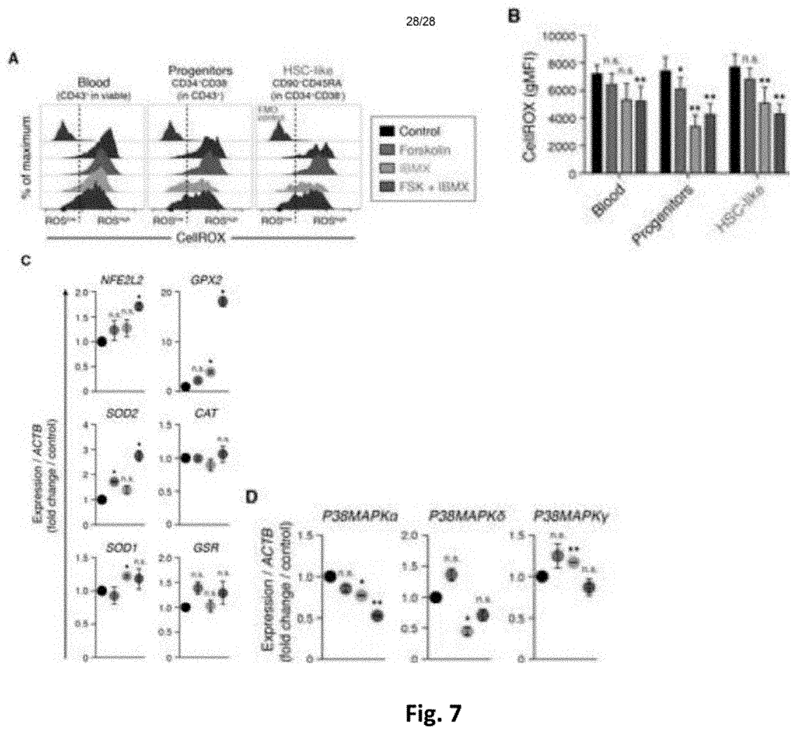

In particular embodiments, activators of the cAMP signaling pathway, such as Forskolin and IBMX may be added to the culture. cAMP activation has been shown to reduce intracellular levels of ROS in pluripotent stem cell differentiation cultures (see FIG. 7, below). In certain embodiments the concentration of Forskolin may be about 1-50, 5-40, 10-30, 15-25, or about 10 μM or 20 μM. In some embodiments, the concentration of IBMX may be about 10-1000, 50-900, 100-800, 200-700, 300-600, or about 400-500 μM or 500 μM.

In some embodiments, additional components may be utilized in the method described above. For example, Sodium Selenite may be added to the culture at a concentration of about 10-200 nM, 50-150 nM, or about 100 nM. As a further example, Butein may be added to the culture at a concentration of about 1-20 μM, 5-15 μM, or about 10 μM.

### EXAMPLES

The following examples are intended to further describe and illustrate various aspects of the invention, but not to limit, the scope of the invention in any manner, shape, or form, either explicitly or implicitly.

### Experimental Procedure for Examples 1-4

hPSCs were routinely maintained as colonies on Murine Embryonic Fibroblasts (MEFs) (Merck Millipore, Darmstadt, Germany) until start of differentiation. Additionally, the hPSCs were cultured with 3 μM CHIR99021 (R&D Systems, McKinley Place, Minn., U.S.A.), during a 48 hour period before start of differentiation, to prime them for mesodermal commitment. Cell lines used were human ES cell lines H1, HUES 2, and HUES 3 (obtained from WiCell, Madison, Wis., and Harvard University, respectively, under material transfer agreements), and the iPS cell line RB9-CB1 (derived from cord blood endothelial cells transduced with tet-inducible lentiviral vectors expressing OCT4, SOX2, LIN28, KLF4, C-MYC). All pluripotent cell lines were karyotypically normal and have earlier been demonstrated to be pluripotent by in vivo teratoma assay and polymerase chain reaction (PCR). Mycoplasma testing was performed routinely to assure that all lines were free of contamination. Cell media used for differentiation of pluripotent stem cells towards blood was MesoTotal (Primorigen Biosciences, Madison, Wis., U.S.A.). At day 0 of differentiation colonies of hPSCs were separated from MEFs by incubation with 0.5 mg/ml Dispase (Invitrogen) in DMEM at 37° C. for 30 minutes.

Detached colonies were collected, washed, and resuspended in MesoTotal before being placed into Corning Costar Ultra-low attachment Plates/Dishes (Sigma-Aldrich, St. Louis, Mo., U.S.A.) after which the colonies formed Embryoid Bodies (EBs) over night. On day 1 and 2 of differentiation the EBs were collected, washed, and received full media changes. On day 4 and 6 50% of the used MesoTotal was replaced with fresh media. On day 8 the EBs were collected and resuspended in 100% fresh MesoTotal before being distributed into wells coated with Matrigel (STEMCELL Technologies, Vancouver, BC, Canada), after which the EBs attached and layers of cells began to spread. On day 10, 12, and 14 100% of MesoTotal was replaced. On day 15 only 90% of the media was replaced to limit loss of free-floating hematopoietic progenitors. On day 16 the cultures were harvested for analysis. All media was collected before the differentiated cells were washed with PBS, singularized using TrypLE (Thermo Fisher Scientific), passed through a 21G needle, and filtered using 30 μm sterile Cup Filicons (BD Biosciences). The cells were then centrifuged at 350G for 8 minutes before being resuspended and counted. Growth media used for sub-culture of CD34-enriched cord blood cells was standardized cord blood expansion media SFEM (STEMCELL Technologies), supplemented with 100 ng/ml each of the following cytokines; hTPO, hSCF, and hFLT3 (all from PeproTech, Rocky Hill, N.J., U.S.A.). The following factors were added to the media if indicated: L-Ascorbic acid (Sigma, St. Louis, Mo., U.S.A.) at 0.378 mg/ml, and Citric acid monohydrate (ACROS Organics/Thermo Fisher, New Jersey, U.S.A.) at 0.158 mg/ml. Also Myeloperoxidase blocker 4-Aminobenzoic acid (Sigma) at 100 μM, and p38 MAPK inhibitor LY2228820 (Selleckchem, Houston, Tex., U.S.A.) at 500 nM, were dissolved in DMSO (Sigma) before addition to media. DMSO was used as vehicle control.

All cells were cultured at 37° C. at 5% CO2. Oxygen levels were either at atmospheric concentration (21%) or kept at 4% by use of a hypoxic incubator (BINDER GmbH, Tuttlingen, Germany). All media was allowed to pre-incubate for 8 hours before addition to cell cultures.

Differentiated cells were washed in PBS before singularization using TrypLE (Thermo Fisher Scientific), passed through a 21G needle, and filtered using 30 μm sterile Cup Filicons (BD Biosciences). Cells were treated with 7AAD to exclude dead cells. Cells were stained using the following anti-human antibodies; fluorescein isothiocyanate (FITC)-conjugated CD45 (eBioscience, 11-0459-42) and CD43 (BD Biosciences, 555475), phycoerythrin-cyanine (PE-Cy7)-conjugated CD34 (Biolegend, 343516), Phycoerythrin (PE)-conjugated CD90 (Biolegend, 328110). CellROX Deep Red Reagent (Thermo Fisher Scientific, C10422), used to detect oxidative stress, was applied according to the manufacturers instruction. Cells were incubated with CellROX Deep Red Reagent, and additional antibodies, for 20 minutes at 37° C. in the dark. Cells were acquired on a FACS LSR II (BD Biosciences) or sorted using a FACS Aria III (BD Biosciences). Analysis was done using FlowJo, version X.0.7 (FLOWJO LLC, Ashland, Oreg., U.S.A.). All FACS gates are based on fluorescence minus one (FMO) controls unless stated otherwise.

Hematopoietic progenitors were sorted and plated with 1.5 ml of MethoCult H4435 (STEMCELL Technologies) into individual wells on Falcon Tissue Culture six-well plates (Thermo Fisher Scientific) at a ratio of 500 cells per well. No additional cytokines or compounds were added to the methylcellulose. Cultures were incubated for 14 days in a standard humidified incubator at 37° C. with 5% CO2. Colonies were counted, and scored by size, using bright-field microscopy.

Hematopoietic progenitors were sorted into ice-cold PBS and cast into 40° C. 1% low melt point agarose (LONZA, Rockland, Me., U.S.A.) on Microscope Slides (Thermo Fisher Scientific). Cells were lysed in NaOH solution at pH>13 over night at 4° C. in the dark. The slides were then rinsed twice with NaOH, at pH 12.3, followed by submersion in a EASYCAST B1 gel runner (Thermo Fisher Scientific) filled with the same solution and allowed to run for 25 minutes at 20V, 50 mA (0.6 V/cm). The slides were then washed with distilled H2O followed by a 5 minutes immersion in 70% EtOH before being allowed to air-dry for 15 minutes. The slides were submerged in TE-Buffer solution with 1:10000X SYBR Green I (Invitrogen, Eugene, Oreg., U.S.A.) and incubated in the dark for 30 minutes, followed by H2O rinsing, and 15 minutes of air-drying. The slides were then immediately evaluated using an IX70 microscope (OLYMPUS, Shinjuku, Tokyo, Japan), equipped with a DP72 camera (OLYMPUS), and images were captured using the software cellSens Standard 1.6 (OLYMPUS). Brightness and contrast adjustments were un-biasedly carried out for all images using Photoshop CS6 (Adobe Systems Inc., San Jose, Calif., U.S.A.) prior to image analysis. Automatic comet analysis and OTM scoring was performed using the previously published ImageJ plug-in OpenComet 29 for ImageJ (version 1.48). This protocol is a modified version of an alkaline Comet Assay protocol published previously.

HSC-like cells were sorted into the following media; IMDM (+Hepes, −Glutamine) (GE Healthcare Bio-Sciences, Little Chalfont, UK), 20% heat-inactivated FBS (Thermo Fisher Scientific), 1X L-Glutamine (Thermo Fisher Scientific), 10 μg/ml Penicillin-Streptomycin Solution (GE Healthcare BioSciences), supplemented with the following cytokines; hSCF, hFLT3, hIL3, hTPO, hGM-CSF (all from PeproTech) at the final concentration of 10 ng/ml. Cells were distributed into wells on Nunc MiniTrays (Thermo Fisher Scientific) at 4 cells per well and 20 μl media. Plates were then placed on elevations in sterile plastic boxes containing PBS to prevent media evaporation. Each well was scored for cell growth at 18 hours, 5 days, 9 days, and 13 days post-sort by image capturing using bright-field microscopy followed by manual unbiased area coverage estimation.

Statistical analysis was, unless stated otherwise, performed using unpaired Student's t test, and the results were considered to be statistically significant at p value<0.05. All graphs depict mean±SEM. The number of biological replicates is indicated by the n value. The graphs and statistical evaluation were done using GraphPad Prism (GraphPad Software, San Diego, Calif., U.S.A.).

### Example 1

### Elevated Reactive Oxygen Species Levels Correlate with the Decreased Function of hPSC-Derived Hematopoietic Progenitors

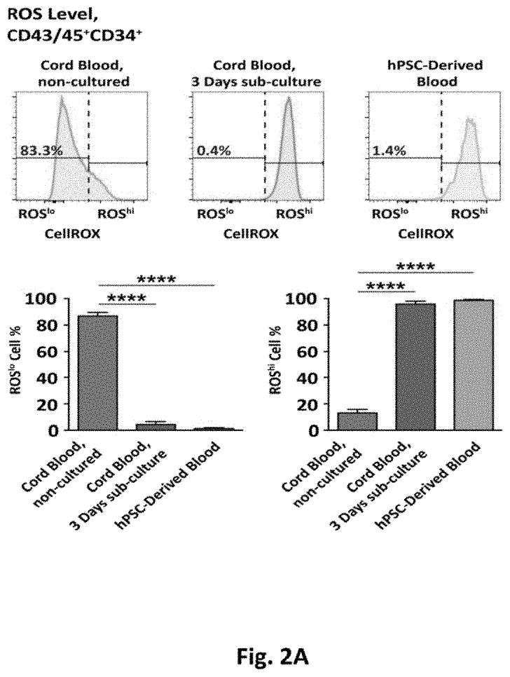

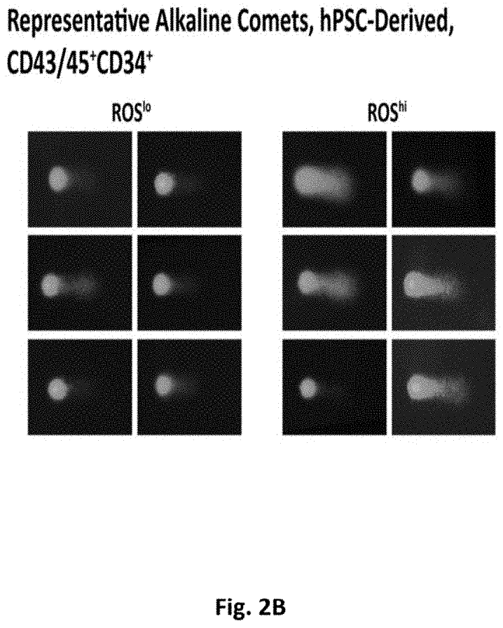

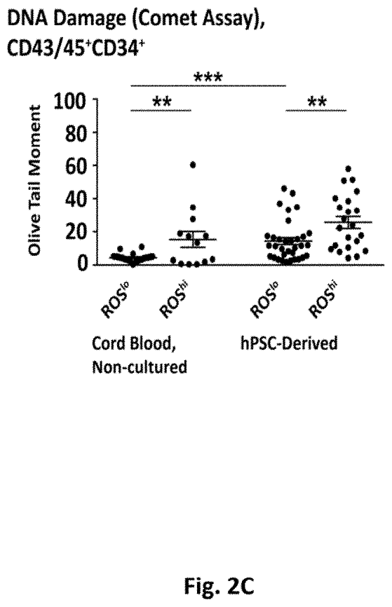

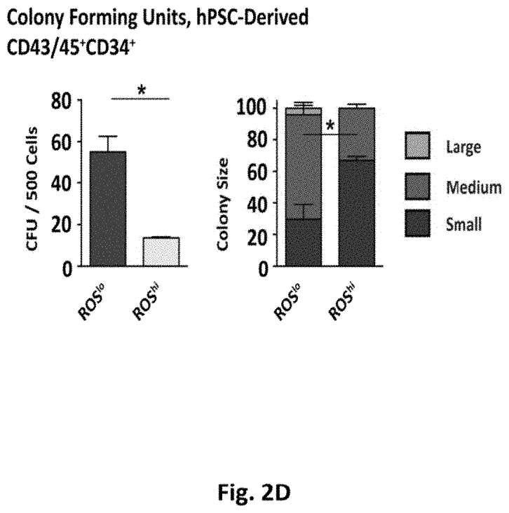

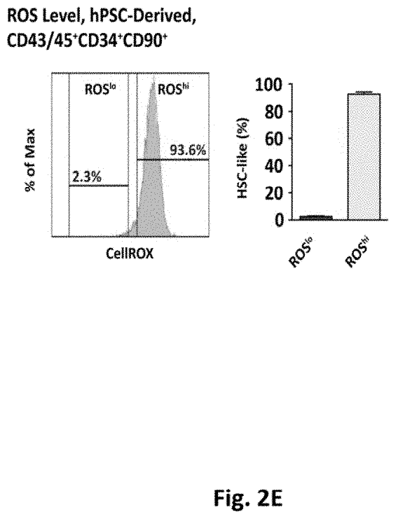

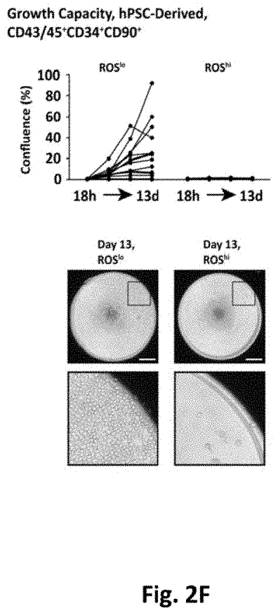

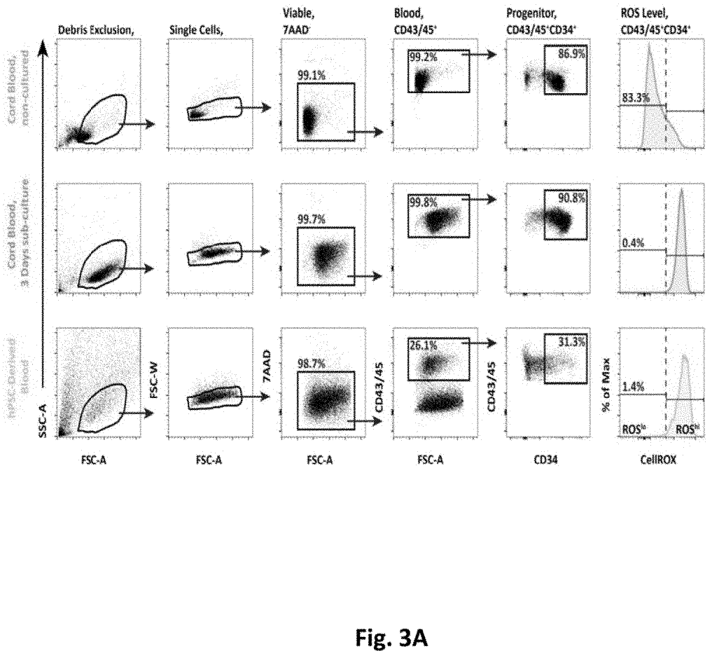

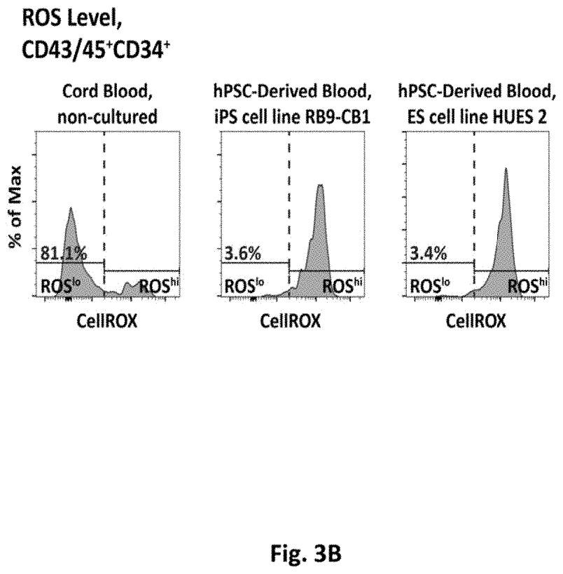

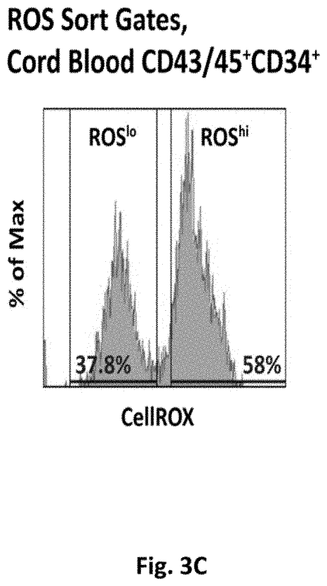

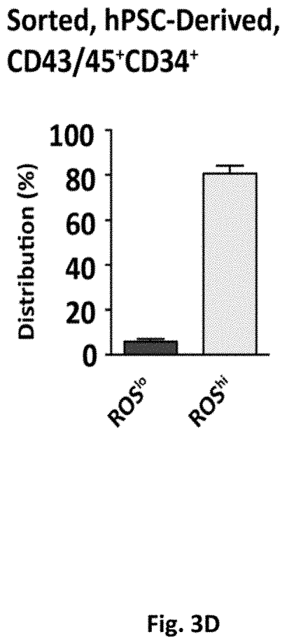

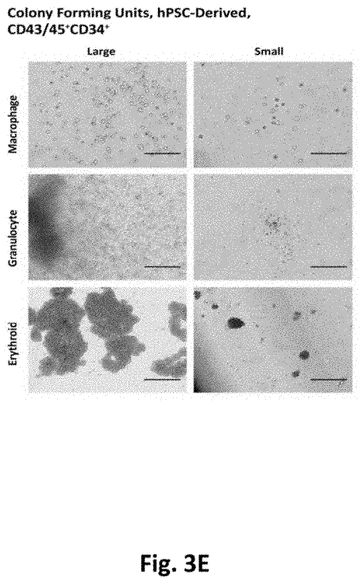

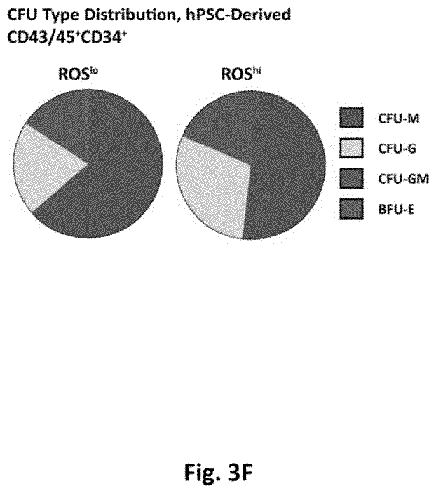

FIG. 2A shows flow cytometry histograms displaying the ROS levels of the hematopoietic progenitor fraction (CD43/45+CD34+) for non-cultured cord blood, 3 days sub-cultured cord blood, and hPSC-derived blood. The gating strategy is detailed in FIG. 3A. Lower panel bar graphs show the ROSlo and ROShi status of hematopoietic progenitors (n=3). FIG. 2B shows representative alkaline comets of ROSlo and ROShi hPSC-derived hematopoietic progenitors. FIG. 2C shows levels of DNA damage in ROSlo and ROShi hematopoietic progenitors, indicated by Olive Tail Moment (OTM), for non-cultured cord blood, and hPSC-derived hematopoietic progenitors (n=3). FIG. 2D is a graph depicting the number of CFU obtained from 500 ROSlo or ROShi hPSC-derived hematopoietic progenitors. The right panel shows the size distribution of the CFUs as indicated. FIG. 2E shows representative FACS sort gates for the ROSlo and ROShi fractions of the hPSC-derived CD43/45+CD34+CD90+ hematopoietic progenitor population. The bar graph displays the distribution of ROSlo or ROShi cells (n=3). FIG. 2F is a graph showing the growth capacity of hPSC-derived CD43/45+CD34+CD90+ cells. The upper panel shows the growth kinetics of a representative sample group (ROSlo and ROShi hPSC-derived CD43/45+CD34+CD90+ cells) indicated by well confluency at 18 hours, 5 days, 9 days, and 13 days after seeding. The lower panel shows representative culture wells, initially seeded with ROSlo or ROShi cells, after 13 days of sub-culture (scale bars=200 μm). The data represents the mean ±SEM. Asterisks indicate significant differences (*p<0.05, **p<0.01, ***p<0.001, ****p<0.0001, n.s., not significant).

### Colony Forming Capacity of ROSlo and ROShi hPSC-Derived Hematopoietic Progenitors

FIG. 3A shows representative flow cytometry dot plots showing hematopoietic cells in non-cultured cord blood, sub-cultured cord blood (3 days), and hPSC-derived blood cells, for the total hematopoietic fraction (CD43/45+), and the hematopoietic progenitors fraction (CD43/45+CD34+). Gates are based on FMO controls, and doublet exclusion. The ROS level for the hematopoietic progenitor fraction is displayed. FIG. 3B shows flow cytometry histograms displaying the ROS level of hematopoietic progenitors from non-cultured cord blood, derived from iPS cell line RB9-CB1, and derived from ES line HUES 2. FIG. 3C shows representative sort gates for ROSlo and ROShi hematopoietic progenitors based on a cord blood sample. FIG. 3D is a bar graph that shows the distribution of sorted ROSlo and ROShi hPSC-derived hematopoietic progenitors (n=3). FIG. 3E contains micrographs of representative CFUs as based on colony type, and size (scale bars=100 μm). FIG. 3F depicts pie charts showing the colony type distribution for CFU-M, CFU-G, CFU-GM, and BFU-E (n=2-3). Data is represented as the mean±SEM.

As described herein this section and elsewhere in the specification, elevated ROS has been shown to impair the function of both murine and human HSCs, and progenitors, and since these primary cells, when cultured in standard in vitro culture conditions, rapidly shift from a ROSlo to a ROShi state the levels of ROS in a pluripotent stem cell differentiation system were evaluated to determine the impact of ROS on the in vitro generation and functionality of hematopoietic cells. Following 16 days of differentiation from the pluripotent state, hematopoietic cell fractions were identified by their cell surface phenotype using the established markers, as follows: total hematopoietic cell fraction identified as CD43/45+ (combined use of early- & pan-hematopoietic markers) 31, the hematopoietic progenitor fraction as CD43/45+CD34+, and the most primitive hematopoietic fraction, previously described as HSC-like 5, as CD43/45+CD34+CD90+. The surface marker CD38, previously described to negatively enrich for HSCs within the CD34+ fraction 34,35, was not included since we have previously demonstrated that the vast majority of CD43/45+CD34+CD90+ cells generated with our differentiation protocol are negative for CD38 5. The cell permeable dye CellROX Deep Red, becoming fluorescent upon presence of intracellular ROS, was used to measure ROS in all cell populations. In conjunction with our analyses using our hPSC-to-blood protocol, freshly isolated CD34-enriched cord blood cells were used as a reference point to indicate physiological levels of ROS, which we defined as ROSlo (FIG. 2A, upper left panel, and FIG. 3A, upper panel). The same cells cultured for 3 days in a hematopoietic progenitor specified media rapidly increased ROS levels, with few cells displaying the ROSlo phenotype, which we defined as ROShi (FIG. 2A, upper middle panel, and FIG. 3A, middle panel). Analysis of ROS in our hPSC-derived hematopoietic progenitors (CD34+) demonstrated the vast majority having the ROShi phenotype (FIG. 2A, upper right panel, and FIG. 3A, lower panel). Similar levels of ROS were observed in hematopoietic progenitors generated from differing hPSC lines (FIG. 3B).

To evaluate the impact of ROS on the functionality of iPS cell derived hematopoietic progenitors, cells were separated into ROSlo and ROShi fractions (FIG. 3C-D). Since ROS is known to cause DNA damage, the extent of DNA damage in ROSlo and ROShi hematopoietic progenitors was directly evaluated. For this purpose the alkaline Comet assay was used that allows for measurement of both single- and double-strand breaks. Analysis of sorted hematopoietic progenitors demonstrated that the ROShi fraction had significantly higher DNA damage as compared to the ROSlo fraction (FIG. 2B-C). In addition, hPSC-derived hematopoietic progenitors sorted for ROSlo or ROShi were evaluated for their growth and differentiation capacities in the colony forming unit (CFU) assay. CFUs were analyzed in terms of number, size, and type (FIG. 1D, FIG. S1E-F). ROSlo hematopoietic progenitors (CD34+) yielded higher numbers of CFUs as compared to ROShi counterparts (FIG. 1D, left panel), and ROSlo cells generated significantly greater numbers of medium and large sized colonies indicating superior proliferative capacity (FIG. 2D, right panel). The differences in ROS level did not change the frequencies of different colony types, however erythroid colonies, which are known to be more sensitive to ROS 37, were only observed in ROSlo progenitor-derived CFUs (FIG. 3F).

Adult HSCs have been demonstrated to be more sensitive to ROS as compared to the downstream hematopoietic hierarchy 38,39. Given the difficulties in generating HSCs from hPSC differentiation cultures capable of repopulation in a xenotransplant assay, we developed an alternative assay to assess the cells functional properties. To evaluate the impact of ROS hPSC-derived CD43/45+CD34+CD90+ cells were sorted for either the ROSlo or ROShi phenotype (FIG. 2E, left panel) and distributed into Terasaki plates to measure their growth at several time-points over a 13-day period (FIG. 2F, upper panel). While ROShi cells demonstrated severely limited cell growth and displayed a flattened morphology indicative of senescence, the ROSlo cells proliferated and gave rise to large clusters of hematopoietic cells (FIG. 2F, lower panel), indicating that the <3% ROSlo cells (FIG. 2E, right panel) of the total CD90+ progenitor fraction contained all the proliferative potential of these cells generated in the standard condition. These results strongly indicate that ROSlo levels are requisite for the expansion/proliferation of these more primitive hematopoietic cells (a key feature of HSC function). Taken together, high ROS levels in iPS-derived hematopoietic differentiation systems, with its associated increase in DNA damage, significantly impairs the growth capacity of more primitive hematopoietic cells.

### Example 2

### ROS Reducing Strategies Specifically Increase ROSlo Hematopoietic Progenitors and HSC-Like Cells

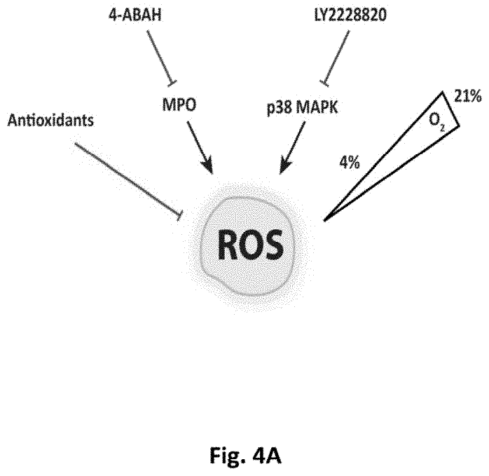

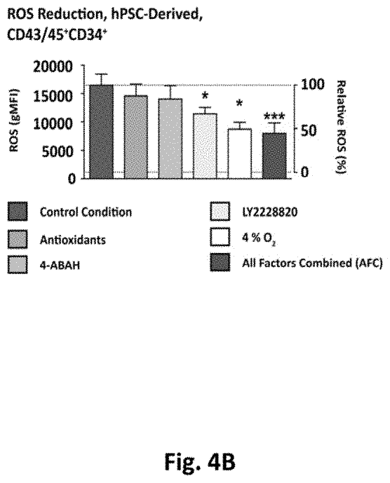

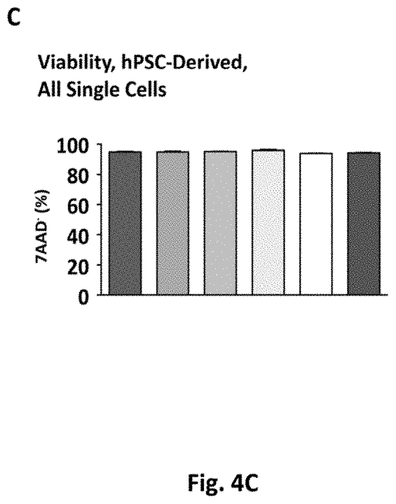

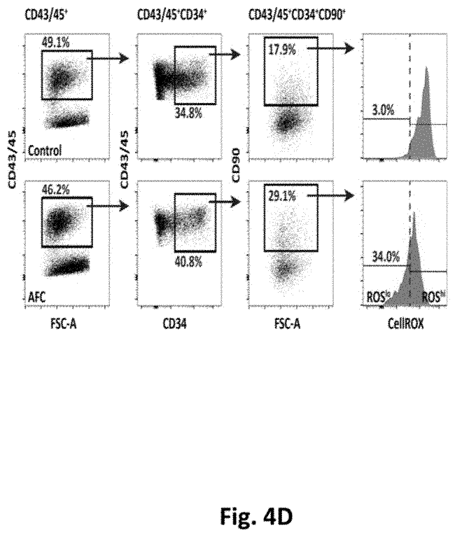

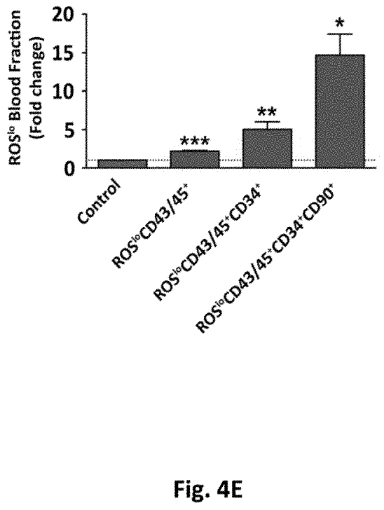

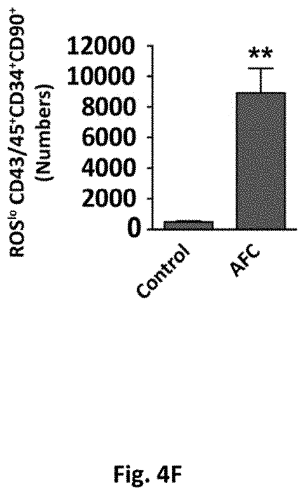

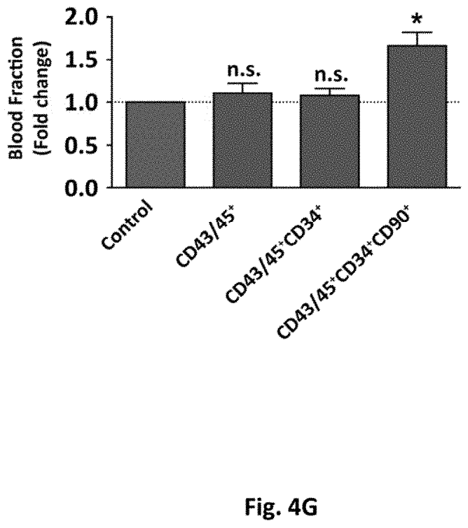

FIG. 4A shows a schematic representation of 4 factors that may be used for ROS reduction in certain embodiments. FIG. 4B is a bar graph showing the ROS (in gMFI) of hPSC-derived hematopoietic progenitors generated with: ROS scavenging by a cocktail of antioxidants (Ascorbic Acid+Citric Acid), myeloperoxidase (MPO) blocking by 4-ABAH, p38 MAPK inhibition by LY2228820, reduction of oxygen tension from 21% to 4%, or All Factors Combined (AFC), and control condition (DMSO) (n=3). Statistics are based on paired parametric t-test. The right y-axis displays extent of ROS reduction as compared to the physiological ROS level of hematopoietic progenitors from non-cultured cord blood (set to 0) and the ROS level of hematopoietic progenitors generated in control conditions (set to 100). FIG. 4C is a bar graph showing the viability (7AAD−) of hPSC-derived cultures generated with the above described conditions (n=3). FIG. 4D shows representative flow cytometry dot plots for hPSC-derived total hematopoietic cells (CD43/45+), hematopoietic progenitors (CD43/45+CD34+), and CD90+ hematopoietic progenitors (CD43/45+CD34+CD90+), generated in control conditions or with AFC. Gates are based on FMO controls. Doublet exclusion and dead cell exclusion (7AAD) were done before applying the gates. The ROS level for the HSC-like fraction is displayed. FIG. 4E is a bar graph showing the fold change in the frequency of ROSlo total hematopoietic cells, ROSlo hematopoietic progenitors, and ROSlo CD90+ hematopoietic progenitors. The values are normalized to control (set as 1) (n=3). FIG. 4F is a bar graph showing the output of ROSlo CD43/45+CD34+CD90+ cells generated from equal amounts of initial hPSCs (n=3). FIG. 4G is a bar graph showing fold change in the frequency of total (ROSlo+ROShi) hematopoietic cells, the hematopoietic progenitor fraction, and the CD90+ hematopoietic progenitor fraction. The values are normalized to control (set as 1) (n=3). Data represent mean±SEM. Asterisks indicate significant differences (*p<0.05, **p<0.01, ***p<0.001, n.s., not significant).

In order to identify means to facilitate a physiological level of ROS in hPSC-derived hematopoietic progenitors, we employed four strategies, each aimed at reducing ROS by a separate mechanism (FIG. 4A): 1) ROS scavenging by a cocktail of antioxidants (Ascorbic Acid and Citric Acid), 2) Myeloperoxidase (MPO) inhibition with 4-aminobenzoic acid (4-ABAH) 22 to prevent innate immune-cell mediated ROS release, 3) p38α Mitogen-Activated Protein Kinase (MAPK) inhibition with the small molecule LY2228820 26 to limit stress-response mediated ROS generation, and 4) reducing ROS by reducing the oxygen concentration to 4% during culture. Applied individually, each of the ROS reducing strategies reduced ROS between 11-47% (FIG. 4B) indicating that active innate immunity, active stress signaling, and accelerated ROS formation due to a supraphysiological oxygen level, collectively contribute to the increased ROS observed in our cells. To provide broad protection from both spontaneous and enzymatically generated ROS, All Factors Combined (AFC) were used, which facilitated the strongest reduction of ROS levels (FIG. 4B), equivalent to a 60% reduction compared to the hPSC-derived control condition and approaching the ROS level of non-cultured cord blood. Importantly, cellular viability was not negatively affected using these ROS reducing strategies (FIG. 4C). However, analysis of the frequencies of total hematopoietic cells (CD43/45+), hematopoietic progenitor cells (CD43/45+CD34+), and cells with the previously described HSC-like surface markers (CD43/45+CD34+CD90+) (FIG. 4D) demonstrated that the AFC significantly increased the frequency of ROSlo total blood cells by 2-fold, and ROSlo progenitors by 5-fold (FIG. 2E). Interestingly, the AFC condition increased the frequency of ROSlo CD43/45+CD34+CD90+ cells by 15-fold (FIG. 4E). Furthermore, from equal amounts of pluripotent starting material, the AFC condition produced 18-fold increase in total output of ROSlo CD43/45+CD34+CD90+ cells compared to the standard condition (FIG. 4F). There was also a modest (1.5-fold) but statistically significant increase in the frequency of the total (ROSlo+ROShi) CD43/45+CD34+CD90+ cells as compared to the standard condition (FIG. 4G) These results together demonstrate that in some embodiments, increased ROS in hematopoietic cells cultured in vitro is contributed by multiple mechanisms, and that a combination of ROS reducing methods allows for significant reduction of ROS, that specifically facilitates increases of ROSlo hematopoietic progenitors and, preferentially, the more primitive CD90+ HSC-like fraction, from hPSCs.

### Example 3

### ROS Reduction Increases ROSlo CD90+ Hematopoietic Cells with Robust Growth Capacity

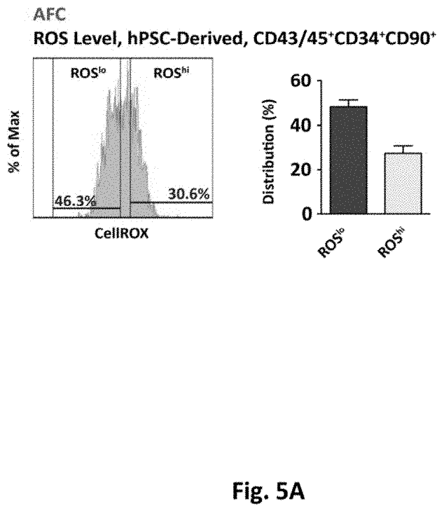

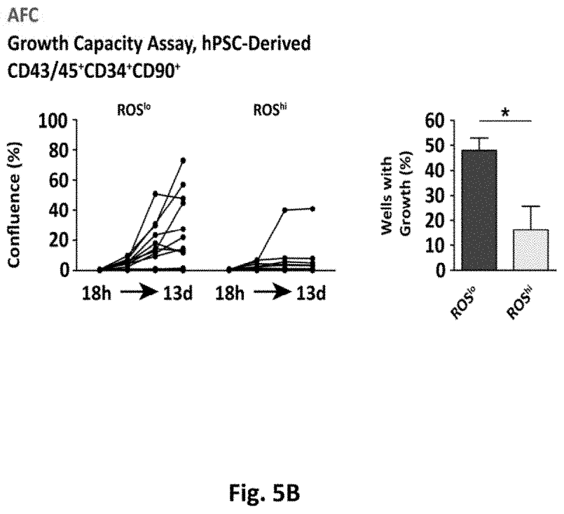

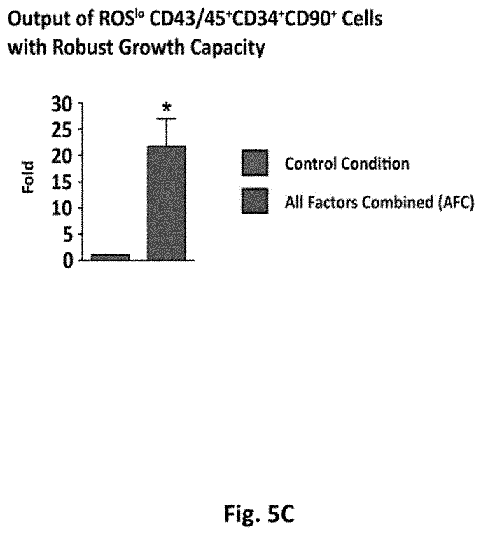

FIG. 5A shows representative FACS gates for sorting the ROSlo and ROShi fractions of CD43/45+CD34+CD90+ cells generated after AFC. Bar graphs show the distribution of ROSlo and ROShi cells (n=3). FIG. 5B shows growth kinetics of the cells, with the left panel showing growth kinetics of a representative sample group (ROSlo and ROShi CD43/45+CD34+CD90+ cells) generated with AFC, as measured by well confluence (%) at 18 hours, 5 days, 9 days, and 13 days post-seeding. The right panel bar graph shows the frequency of wells with >10% well confluence at day 13. FIG. 5C shows the fold change in the generation of ROSlo CD43/45+CD34+CD90+ cells with high proliferative capacity using AFC as normalized to the control condition (DMSO). Data represent mean±SEM. Asterisks indicate significant differences (*p<0.05, n.s., not significant).

To evaluate the function, in terms of growth capacity, of cells generated in the AFC condition, CD43/45+CD34+CD90+ cells were separated into ROSlo and ROShi fractions and were plated into Terasaki wells. Sort-gates were based on the ROS profile of a cord blood sample simultaneously analyzed. Approximately 50% of these hPSC-derived cells displayed a ROSlo phenotype (FIG. 5A). ROSlo CD43/45+CD34+CD90+ cells generated with the AFC demonstrated higher growth capacity compared to the limited performance of the ROShi fraction (FIG. 5B, left panel). The proportion of ROSlo cells capable of proliferation was also increased compared to the ROShi, thus confirming that the ROS reducing factors not only reduced ROS, but also increased the functional growth capacity of the CD90+ progenitor fraction (FIG. 3B, right panel). Moreover, the total increase in generated ROSlo CD43/45+CD34+CD90+ cells with robust growth capacity was 22-fold higher with AFC, as compared to the standard condition (FIG. 5C). These findings demonstrate that high ROS levels limit function in terms of proliferation in the most primitive hPSC-derived hematopoietic progenitors , and that our strategies to reduce ROS significantly increase the output of such hematopoietic progenitors with growth capacity.

In this study, we also evaluated the engraftment capacity of cells generated either with our standard condition or with the ROS reducing AFC condition. Using a stringent assessment of engraftment in transplanted NSG mice we saw very low levels of human hematopoietic cells that were not discernable over negative controls, indicating that the 60% reduction of ROS reported in this study was insufficient to endow our hPSC-derived hematopoietic cells with engraftment capacity (data not shown). Nonetheless, the level of reduction achieved demonstrated that a feature of HSC function (cellular expansion) was improved for the hPSC-derived cells under the AFC condition and therefore the in vitro generation of functionally relevant hematopoietic stem cells will likely require ROS level regulation.

### Example 4

### Endothelial Cells Have Low ROS, and ROS Reducing Strategies Specifically Reduce ROS in

More Primitive Hematopoietic Cell Fractions

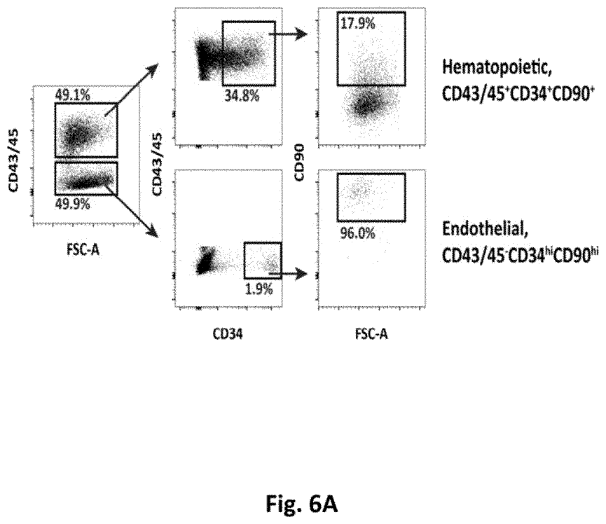

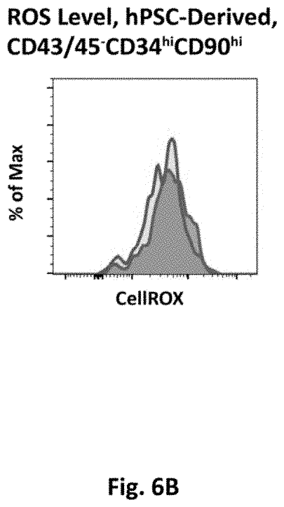

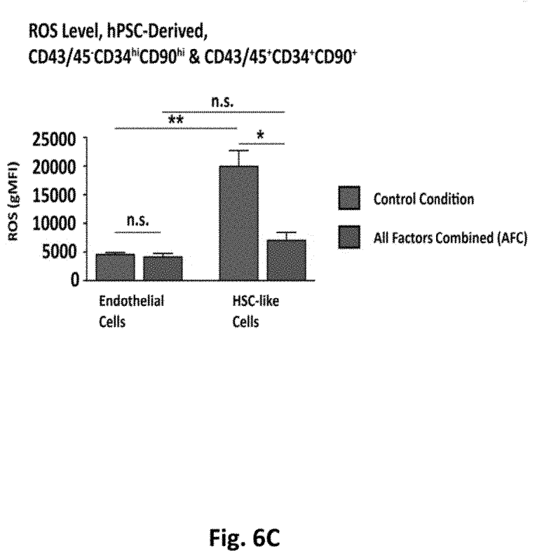

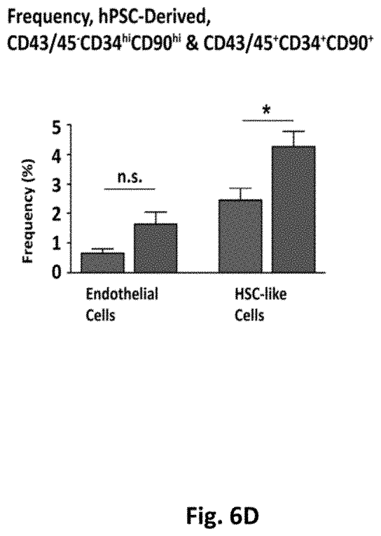

FIG. 6A shows representative flow cytometry dot plots showing hPSC-derived CD90+ hematopoietic progenitor cells (CD43/45+CD34+CD90+) and endothelial cells (CD43/45-CD34hiCD90hi). Gates are based on FMO controls, doublet exclusion, and dead cell exclusion. FIG. 6B shows a representative histogram plot displaying the ROS levels of the endothelial population for the DMSO control (gray) and the AFC (green). FIG. 6C shows the ROS (gMFI) of the endothelial and the CD90+ hematopoietic progenitor fractions generated with control condition (DMSO) and AFC (n=3). FIG. 6D shows the frequency of the endothelial and the CD90+ hematopoietic progenitor fractions, generated with control conditions and AFC (n=4). Data represents mean±SEM. Asterisks indicate significant differences (*p<0.05, **p<0.01, n.s., not significant).

The origin of hematopoietic cells both in vivo during embryonic development and in vitro during differentiation from pluripotent stem cells has been shown to occur from endothelial cells with hemogenic potential. To evaluate the levels of ROS in other cell types present in the blood directed differentiation cultures, the ROS levels in the CD43-CD34hiCD90hi endothelial cell fraction was evaluated (FIG. 6A-B). The intracellular ROS levels in endothelial cells derived from iPS using the standard culture condition were uniformly low, and were not further reduced with the addition of the AFC (FIG. 6C). This is in contrast to the CD43/45+CD34+CD90+ fraction of hematopoietic cells in the same wells, where AFC facilitated a significant reduction of intracellular ROS as compared to the standard condition. Moreover, while the AFC did not significantly change the frequency of the endothelial cells (FIG. 6D), a trend suggested a change in frequency similar to the 1.5-fold increase of the CD43/45+CD34+CD90+ HSC-like fraction. These observations suggest that there are large differences in the abilities of differing cell types in the differentiation system to regulate their ROS levels; with the broad endothelial cell fraction effectively managing its intracellular ROS levels, while hematopoietic progenitors are unable to retain a low ROS profile in culture, and displaying deficits in growth potential as a result. In comparison with CD34+ progenitors, the CD90+ hematopoietic progenitor fraction, saw the greatest reduction in ROS levels using AFC, yielding the greatest increases in frequencies and numbers of cells, and resulted in a 21 fold increase in cells with growth capacity, together suggests that (FIG. 2E), indicates that the most primitive hematopoietic cells in the culture system may be the most sensitive to ROS and have the most to gain from ROS reduction strategies.

FIGS. 7A-D depicts embodiments of the results of using a method to reduce intracellular levels of ROS by cAMP activation in pluripotent stem cell differentiation cultures. Cyclic AMP induction may reduce oxidative stress and induces CXCR4 in hPSC-derived hematopoietic cells. Part A shows flow cytometric analysis for detection of reactive oxygen species (ROS) in differentiated hPSC-to-hematopoietic cells at day 14 of differentiation. Representative flow cytometry plots (biexponential x-axis) show ROS levels in the hematopoietic surface phenotypes. FMO control, fluorescence minus-one (staining control). Part B shows quantification of geometric mean fluorescence intensity (gMFI) of CellROX dye as indicated in (A). Data represents mean±S.E.M. of three independent experiments. Statistical analysis was performed using the t-test. Significance is shown compared to the control setting. *, p<0.05, **, p<0.01, n.s., not significant. Part C shows qRT-PCR expression analysis of the indicated redox state-regulating genesin PSC-derived hematopoietic cells. Relative expression of each gene to housekeeping gene ACTB (β-ACTIN) was calculated and mean fold change respective to control condition (set at one) is shown. Data represents mean±S.E.M. of two independent experiments. Statistical analysis was performed using the t-test. Significance is shown compared to the control setting. *, p<0.05, **, p<0.01, n.s., not significant. Part D depicts qRT-PCR expression analysis of the indicated p38MAPK-related genes in PSC-derived hematopoietic cells. Relative expression of each gene to housekeeping gene ACTB (β-ACTIN) was calculated and mean fold change respective to control condition (set at one) is shown. Data represents mean±S.E.M. of two independent experiments. Statistical analysis was performed using the t-test. Significance is shown compared to the control setting. *, p<0.05, **, p<0.01, n.s., not significant.

Although this disclosure describes certain embodiments, it will be understood by those skilled in the art that many aspects of the methods and devices shown and described in the present disclosure may be differently combined and/or modified to form still further embodiments or acceptable examples. All such modifications and variations are intended to be included herein within the scope of this disclosure. Indeed, a wide variety of designs and approaches are possible and are within the scope of this disclosure. No feature, structure, or step disclosed herein is essential or indispensable. Moreover, while illustrative embodiments have been described herein, the scope of any and all embodiments having equivalent elements, modifications, omissions, combinations (e.g., of aspects across various embodiments), substitutions, adaptations and/or alterations as would be appreciated by those in the art based on the present disclosure. While certain embodiments have been described, these embodiments have been presented by way of example only, and are not intended to limit the scope of protection.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of mediating cellular stress during transition of a cell into another cell type, comprising:
culturing said cell under conditions that lead to said transition of said cell while carrying out two or more of the following:
reducing the available oxygen in a medium surrounding the cell;
treating the cell with an antioxidant;
inhibiting a cellular stress response pathway of the cell;
inhibiting an innate immune response of the cell; and
activating a cellular pathway that reduces the concentration of intracellular reactive oxygen species.

2. A method of mediating cellular stress in a human blood precursor cell during transition of said precursor cell into a blood cell, comprising:
culturing said human blood precursor cell under conditions that lead to said transition of said precursor cell into a blood cell while carrying out one or more of the following:
reducing the available oxygen in a medium surrounding the human blood precursor cell;
treating the human blood precursor cell with an antioxidant;
inhibiting a cellular stress response pathway of the human blood precursor cell;
inhibiting an innate immune response of the human blood precursor cell; and
activating a cellular pathway that reduces the concentration of intracellular reactive oxygen species.

3. The method of claim 1 or claim 2, wherein said transition comprises differentiation of the cell to a more committed cell type.

4. The method of claim 1 or claim 2, wherein said transition comprises a conversion of the cell into an induced pluripotent stem cell.

5. The method of claim 1 or claim 2, wherein activating a cellular pathway that reduces the concentration of intracellular reactive oxygen species comprises treating the cell with a cAMP signaling activator.

6. The method of claim 5, wherein the cAMP signaling activator comprises Forskolin or IBMX.

7. The method of claim 1 or claim 2, wherein reducing the available oxygen in a medium surrounding the cell comprises placing the cell in a hypoxic environment.

8. The method of claim 1 or claim 2, wherein the antioxidant comprises a component selected from the group consisting of ascorbic acid, citric acid, vitamin E, selenium, melatonin, NAC, glutathione, thioredoxin, nicotinamide adenine dinucleotide phosphate, Superoxide dismutase, Catalase, and Glutathione peroxidase, and Citric acid monohydrate.

9. The method of claim 8, wherein the concentration of ascorbic acid is about 0.011-0.55 mg/ml.

10. The method of claim 8, wherein the concentration of citric acid is about 0.115-1.15 mg/ml.

11. The method of claim 1 or claim 2, wherein inhibiting the cellular stress response pathway comprises inhibiting mitochondrial p53 mediated apoptosis.

12. The method of claim 1 or claim 2, wherein inhibiting the cellular stress response pathway comprises inhibiting p38 mediated senescence.

13. The method of claim 10, wherein inhibition of the p38 mediated senescence comprises treating the cell with LY2228820 at a concentration range of about 20-500 nM.

14. The method of claim 1 or claim 2, wherein inhibiting the cellular stress response pathway comprises inhibiting endoplasmic reticulum stress.

15. The method of claim 1 or claim 2, wherein inhibiting the cellular stress response pathway comprises inhibiting non-mitochondrial calpain mediated stress.

16. The method of claim 1 or claim 2, wherein inhibiting the innate immune response comprises inhibiting myeloperoxidase production with a myeloperoxidase inhibitor.

17. The method of claim 16, wherein the myeloperoxidase inhibitor comprises 4-ABAH at a concentration range of about 100 μM.

18. The method of claim 11, wherein inhibiting the cellular stress response pathway comprises inhibiting mitochondrial p53 mediated apoptosis with pfilthrin-μ.

19. The method of claim 1 or claim 2, wherein inhibiting the cellular stress response pathway comprises inhibiting non-mitochondrial calpain mediated apoptosis of the cell.

20. The method of claim 19, wherein inhibiting non-mitochondrial calpain mediated apoptosis of the cell comprises treating the cell with MDL28170 at a concentration range of about 0.5-25 μM.

21. The method of claim 1 or claim 2, wherein reducing the available oxygen for a cell comprises reducing the amount of oxygen in a culture system to about 4%.

22. The method of claim 1 or claim 2, wherein the cell is an induced pluripotent stem (iPS) cell.

23. A medium for the de novo generation of human blood cells, comprising two or more of the following:
an antioxidant;
an inhibitor of mitochondrial p53 mediated apoptosis;
an inhibitor of non-mitochondrial calpain mediated apoptosis;
an inhibitor of endoplasmic reticulum stress;
an innate immune response inhibitor;
an inhibitor of p38 mediated senescence; and
a cAMP signaling pathway activator.
